(19) United States
(12) Patent Application Publication
(10) Pub. No.: US 2006/0294609 A1
(43) Pub. Date: Dec. 28, 2006

(54) Production of cloned offspring from cooled carcasses

(76) Inventors: Steven L. Stice, Athens, GA (US); John Gibbons, Clemson, SC (US); Donald Respess, Lakeland, FL (US)

Correspondence Address: KING & SPALDING LLP, 1180 PEACHTREE STREET, ATLANTA, GA, 30309, US

(21) Appl. No.: 10/512,153

(22) PCT Filed: Apr. 24, 2003
(86) PCT No.: PCT/US03/13088

§ 371 Date: May 25, 2006

Related U.S. Application Data
(60) Provisional application No. 60/375,179, filed on Apr. 24, 2002.

(51) Int. Cl. A01K 67/027

(52) U.S. Cl. 800/15; 800/17

(57) ABSTRACT

Genetic material is derived from animals post-mortem, and used in nuclear transfer processes to produce cloned embryos and live cloned animals having genetic make-ups identical to the post mortem animals. The method has particular applicability to the management and breeding of livestock, to the production of animals having desired genetic traits, and to the integration of those genetic traits into selective breeding operations.

### FIELD OF THE INVENTION

This invention is in the field of animal production, and relates particularly to the use of animal cloning procedures to improve the overall genetic makeup of breeding stock and herds of domesticated livestock for human use and consumption.

### BACKGROUND OF TH INVENTION

Genetics and selective breeding have played a role in animal husbandry throughout the centuries. In one oft-repeated instance of selective breeding reported in the 30th Chapter of Genesis, the biblical patriarch Jacob employed selective breeding between the sheep of his flock and the sheep of a flock owned by his father-in-law Laban to greatly increase the number and hardiness of sheep in his flock at the expense of his father-in-law.

Until the last several years, however, the genetics of livestock herds have generally been improved simply by the natural or artificial insemination of female breeding stock by genetically superior male studs. While this process certainly improves the genetic makeup of bred herds, it does so slowly, and without a high degree of consistency or certainty. The genetic quality of the offspring cannot be guaranteed because the offspring inherits the genetic characteristics of both parents and there is no way to predict the dominance of genetic characteristics imparted by the parents. Moreover, while more sophisticated breeding operations have integrated female genetics into their breeding programs, most breeding operations, especially for cattle, have relied simply upon integrating the superior genetics of males into the herd. Insemination has not allowed breeders to rapidly propogate genetics from many high-value females, such as superior milk-producing cows, because of the need to remove the cows from profitable economic activity to carry out the breeding program.

Animal cloning techniques pioneered in the last decade have added a new dimension to the breeder's arsenal of strategies for improving herd genetics. By cloning select animals and integrating cloned animals into the breeding pool, the breeder can decrease the generational lag required before observing measurable herd improvements in succeeding generations of animals. In addition, the breeder can clone female and male animals with similar ease, and can thus readily integrate the genetics of superior female animals into livestock herds.

Despite these improvements, existing cloning techniques still suffer from several drawbacks. One of the primary drawbacks is the need to accurately identify the animals that should be cloned for selective breeding. This is especially true when one wishes to clone based upon desirable meat characteristics, because the animal cannot be identified until after it has been slaughtered, cooled, and graded at the slaughterhouse. Cloning has not been an available option to breeders in this situation, or in any situation in which the animal sought to be cloned has already died.

In addition, in many circumstances it is simply impractical to identify desirable genetic characteristics until after an animal has been slaughtered. For example, it is difficult if not impossible to obtain tissue samples from large animal populations to identify rare genetic traits, or to identify unique combinations of genetic traits in such animals. This is especially true when herds sought to be screened are owned by multiple parties. The slaughterhouse offers a centralized location for tissue sampling and screening that does not present the ownership issues associated with animal herds, and that does not involve the difficulties associated with collecting tissue samples from live animals. Once again, however, cloning has not been an available option to breeders in situations where the animal sought to be cloned is either sterile or has already died.

### OBJECTS OF THE INVENTION

It is an object of the invention to improve the options available to animal breeders for improving the genetic makeup of breeding stock and livestock.

It is another object of the invention to clone animals based upon characteristics in the animal observed post-mortem, such as meat quality scored at a slaughterhouse.

Still another object is to integrate the genetics of animals cloned from tissue samples derived post-mortem into breeding stock and livestock of livestock producers.

It is a further object of the present invention to facilitate the widespread screening of livestock for desirable genetic traits, and cloning of animals based upon those genetic traits.

It is still another object of the invention to enable the use of post-mortem tissue in cloning operations, to propogate the genetics of a deceased animal, and to reintroduce those genetics to a breeding herd.

It is a further object of the present invention to increase the uses of nuclear transfer in xenographic and other transgenic applications of nuclear transfer technology.

Still a further object is to increase the uses of nuclear transfer in the development of stem cells for therapeutic and investigational purposes.

### OVERVIEW OF THE INVENTION

The inventors have surprisingly discovered that genetic material can be derived from animals post-mortem, and that such genetic material can be used in nuclear transfer processes to produce cloned embryos and live cloned animals having genetic make-ups identical to the post-mortem animals. The inventors' discovery has particular applicability to the management and improvement of breeding livestock, to the production of animals having desired genetic traits, and to the integration of those genetic traits into selective breeding operations.

Therefore, in its broadest sense the invention provides a method of producing a cloned or genetically modified non-human embryo comprising:
a) transferring DNA from a donor cell derived from post-mortem non-human mammalian tissue to an oocyte to form a nuclear transfer unit; and
b) culturing said nuclear transfer unit to establish a nuclear transfer embryo.

In a preferred embodiment, the embryo thus produced is transferred into a recipient female so as to produce a fetus that undergoes full fetal development and parturition to generate a live-born animal.

The ability to clone livestock using post-mortem tissue presents a host of opportunities for improving the efficiency of animal livestock operations. One of the principal advantages relates to the fact that the value of many animals, particularly animals that are raised and sacrificed for their meat value, is not known until after the animals have been sacrificed and the quality of meat can be assessed. For example, it is common in the beef industry to grade a carcass for quality within about 40-48 hours of animal slaughter, typically after the carcass has been cooled to about 0° C. By using this technology, carcasses that exhibit desired qualities can be preferentially selected for cloning and subsequent integration of the cloned offspring into animal breeding operations.

Because the tissue employed in the present invention is derived post-mortem, another substantial advantage of the present invention is that the tissue from which the donor cells for nuclear transfer are derived is not necessarily the same as the tissue in which the desirable characteristics are observed. Thus, it is now possible to employ donor cells that might improve developmental efficiencies associated with the cloning procedure, such as cells derived from reproductive tract tissue, that as a practical matter are not available for use before death.

Still another principal advantage of using post-mortem tissue to clone livestock is the breadth and depth of the impact that such cloning operations promise to have on an animal rearing operation. Whereas cloning has conventionally been employed to replicate only a few of the highest value animals in an animal herd (typically stud bulls that can most rapidly propagate desired genetic characteristics), cloning based upon meat quality acts more broadly as a platform for improving breeding operations because of the higher number of animals that are cloned to achieve significant genetic improvements to animal herds. Although higher numbers of animals are cloned to achieve these effects, the herd eventually benefits because (1) the desired genetics from the cloned animals are more widely spread, and (2) the desired genetics are propagated through males and females, which in the past have diluted genetic concentration efforts, and (3) the animals in the breeding herd with lower genetic quality can be removed and replaced within the breeding system. Thus, in another embodiment the invention provides a livestock breeding operation that comprises a plurality of cloned breeding animals, wherein the cloned animals are derived from nuclear transfer cloning employing post-mortem animal tissue.

Still another advantage of the present invention relates to the sheer quantities of animal tissue available post-mortem, and the ability to readily screen those tissues for desired genetic characteristics. For example, it is becoming common for livestock owners to screen their herds for selected genetic characteristics, such as polymorphisms in genes that produce desirable meat characteristics. Animals that display such polymorphisms or genetic characteristics could then conceivably be beneficially clonally propagated. Until now, however, only live animals have been screened for these genetic traits because it has been thought that only live animals could be cloned. The ability to clone based on post-mortem tissue opens up entirely new possibilities for genetic screening. Screens are no longer limited to particular herds whose owners have consented to such screens, but can instead be performed using animal populations from multiple herds that have been consolidated through a slaughterhouse or other downstream meat processing operation. Thus, in yet another embodiment the invention provides a method of producing a cloned or genetically modified non-human embryo comprising:
a) providing two or more post-mortem tissue samples from two or more different animals;
b) screening the samples for pre-selected physical, genetic and/or phenotypic traits;
c) transferring DNA from one or more donor cells derived from one or more of the animals to an oocyte to form a nuclear transfer unit; and
d) culturing said nuclear transfer unit to establish a nuclear transfer embryo.

### ADDITIONAL DISCUSSION

As mentioned, in one particular embodiment the invention provides a method of producing a cloned or genetically modified non-human mammalian embryo comprising:
a) transferring DNA from a donor cell derived from post-mortem non-human mammalian tissue to an oocyte to form a nuclear transfer unit; and
b) culturing said nuclear transfer unit to establish said embryo.

In a particularly preferred embodiment, the method further comprises transferring said embryo into a recipient female so as to produce a fetus that undergoes fall fetal development and parturition to generate a live-born animal.

It will be understood that the invention can be practiced with any mammalian species. The term “mammalian” as used herein refers to any animal of the class Mammalia. The class Mammalia further includes canid (any animal of the family Canidae, including a wolf, a jackal, a fox, or a domestic dog), felid (any animal of the family Felidae, including a lion, a tiger, a leopard, a cheetah, a cougar, or a domestic cat), murid (any animal of the family Muridae including a mouse or a rat), leporid (any animal of the family Leporidae including a rabbit), ursid (any animal of the family Ursidae including a bear), mustelid (any animal of the family Mustelidae including a weasel, a ferret, an otter, a mink, or a skunk), primate (any animal of the Primate order, including an ape, a monkey, a chimpanzee, or a lemur), ungulate (any animal of the polyphyletic group formerly known as the taxon Ungulata, including a camel, a hippopotamus, a horse, a tapir, an elephant, a sheep, a cow, a goat, or a pig), ovid any animal of the family Ovidae (including a sheep), suid any animal of the family Suidae, including a pig or a boar), equid (any animal of the family Equidae, including a zebra an ass, or a horse), bovid (any animal of the family Bovidae, including antelope, an oxen, a cow, a bison, or a goat), caprid (any animal of the family Caprinae, including a goat), and cervid (any animal of the family Cervidae, including a deer). Preferred mammals for practicing the present invention include animals of the class ungulate, ovid, suid, and bovid. Particularly preferred mammalian species for practicing the present invention are bovine and porcine.

The term “post-mortem” refers to tissue derived from an animal that has died, i.e. in which all vital functions have ended without possibility of recovery. The timing of death is not of critical importance to the instant invention. Thus, donor cells employed in the instant invention can be derived from animals that have just died, animals that died at least about 1, 2, 5, 10, 20, or forty hours earlier, and animals that died more than 3 days, 7 days, 30 days, or one year earlier. The foregoing time periods are measured from the time that an animal dies until genetic material from the donor animal is transferred to an oocyte in a nuclear transfer procedure. An upper limit can also be imposed on the foregoing time limits to establish a range that spans 20 years, 10 years, 5 years, 1 year, 60 days, 30 days, 7 days, 3 days, 40 hours or 20 hours.

The post-mortem tissue is preferably cooled immediately upon death or shortly thereafter (i.e. within about 6, 12, or 24 hours). The temperature to which the tissue is cooled is not critical to the invention but generally will be below about 20, 15, 10, 5, 0, or −5° C. A lower limit can be imposed on the foregoing temperature limits to define a range that spans 25, 20, 15, or 10° C.

As mentioned above, the invention is particularly well suited for screening large populations of animals for desirable physical, phenotypic, and/or genetic traits, because of the large numbers of animals that are typically available for sampling in a slaughterhouse or other downstream meat processing or distribution operation. Thus, in another embodiment the invention provides a method of producing a cloned or genetically modified non-human embryo comprising:
a) providing two or more post-mortem tissue samples from two or more different animals;
b) screening the samples for pre-selected physical, genetic and/or phenotypic criteria;
c) transferring DNA from one or more donor cells derived from one or more of the animals to an oocyte to form a nuclear transfer unit; and
d) culturing said nuclear transfer unit to establish said embryo.

In a series of preferred embodiments, more than 4, 10, 25, 50, 100, 250, or even 1000 samples are screened for the selected criteria. An upper limit can be imposed on the foregoing number of samples to define a range that spans 1000, 250, 100, 50, 25, 10, or 4 samples. The samples can be derived from more than 1, 2, 5, 10, or more herds of animals. The term “herd” refers to a group of domesticated animals of one kind kept together under human care or control.

In another preferred embodiment, the tissue sample that is screened is meat intended for human consumption (i.e. muscle tissue and embedded fat). As mentioned above, while the donor cell used in the nuclear transfer operation can be obtained from the tissue sample that is actually screened, it is preferentially derived from other tissues of the selected animal where such tissues demonstrate higher cloning efficiencies. In one preferred embodiment, the tissue is selected from a cattle carcass, and in an even more preferred embodiment the tissue is derived from a kidney.

Tissue can be selected for cloning based upon a number of physical, phenotypic, and/or genetic traits. In one embodiment, the tissue is selected at a slaughterhouse for cloning based upon the meat quality or yield grade assigned to the tissue. For example, it is known that the United States Department of Agriculture (“USDA”) has quality grades for beef, pork, veal, lamb, yearling mutton, and mutton. The beef quality grades are USDA Prime, Choice, Select, Standard, Commercial, Utility, Cutter, and Canner. Similarly, lamb is graded USDA Prime, Choice, Good, Utility and Cull. In addition, USDA has yield grades for beef, pork, and lamb (i.e. yield ratio of lean to waste), ranging from YG1 to YG5, wherein YG1 represents the leanest cut of meat and YG5 the fattest. An animal can be selected for cloning based upon any one of these quality or yield grades, though preferably only meat that is graded Prime or YG1 or with other high commercial values or cost saving opportunities will be selected for cloning. Other physical characteristics that could form the basis of the cloning decision include marbling, rib-eye muscle area, dressing percentage, and meat tenderness.

An animal can also be selected for cloning based upon the presence of desired nutritional characteristics. For example, meat is a source of protein, niacin, vitamins B₆ and B₁₂, iron, phosphorus, and zinc. Conversely, an animal can be cloned based upon its lack of undesirable nutritional characteristics such as fat, saturated fat, and cholesterol, which are also present in all meat. An animal can be selected for cloning based upon any of the foregoing nutritional characteristics, or any combination of these characteristics.

The meat can also be screened for desired genetic characteristics. For example, WO 02/02822 indicates that there are two alleles corresponding to one of the genes responsible for variations in beef tenderness, one that enhances beef tenderness (the t⁺ allele) and one that reduces tenderness (the t⁻ allele). A t⁺t⁺ animal will produce beef that has a lower Warner Bratzler shear force, on average, and therefore is more tender than an animal with a t⁻t⁻ genotype. Similarly, WO 01/92570 describes an assay to identify pigs having a genetic predisposition to musculature with improved meat quality characteristics. Preferred markers are: i) SW413, SW1482, SW439, S0005, SW904 or regions of chromosome 5 spanning therebetween; or ii) SWR68, S0024, SW827, SW727, SW539, or regions of chromosome 9 spanning therebetween; or iii) SW2093, SW2116 or regions of chromosome 9 spanning therebetween. WO 01/75161 describes genetic markers in the porcine melanocortin4 receptor (MC4R) gene which are associated with favorable meat quality traits including, drip loss, marbling, pH and color. WO 00/69882 describes a polymorphism in the CYP11a1 gene which is associated with rate of gain, carcass length, and litter size in various commercial livestock (particularly porcine). WO 94/21681 discloses growth differentiation factor-8 (GDF-8) that is implicated in the formation of muscle mass. Any of the foregoing genotypes can be selected for practicing the present invention.

Use of Cloning to Breed and Concentrate Selective Traits

Still other embodiments pertain to the integration of genetics from cloned livestock into animal herds, and to methods of integrating such genetics into livestock herds. In one embodiment particularly suited for the instant application these methods are practiced employing post-mortem tissue as the source of donor cells. However, it should be understood that the instant methods can similarly be extended to tissue derived from live animals as well.

Thus, in another embodiment the invention provides a method of improving the genotypical, phenotypical, or physical characteristics of a herd of animals comprising:
a) transferring DNA from a donor cell derived from non-human mammalian tissue to an oocyte to form a nuclear transfer unit;
b) culturing said nuclear transfer unit to establish a nuclear transfer embryo;
c) transferring said embryo into a recipient female so as to produce a fetus that undergoes full fetal development and parturition to generate a live-born animal; and
d) mating said live-born animal with one or more animals of the herd.

One of the most promising features of this invention is its ability to integrate cloning of cattle into breeding programs. In the methods of the instant invention, a livestock producer maintains one or more herds of cattle comprising both males and females. One stud bull is provided for propagating the herd, typically at a ratio of about 20-100, 30-70, or 40-60 females per stud. The male and female offspring are generally considered “terminal offspring” and are sold at market upon reaching sufficient maturity. In order to prevent the male offspring from interfering with the breeding process and stud mating, they are typically castrated shortly after birth. Female offspring are generally also not allowed to enter the breeding process, and are typically segregated from the herd in pens and the like to prevent such entry until they are ready for marketing.

A male stud associated with a herd is eventually replaced by another male stud for reasons of age, health, or stud capacity, or to implement a genetic improvement and/or change in the herd's offspring. A male stud generally is part of a herd for about 1-10 years before it is rotated to a different herd or sacrificed for its meat value.

Females are typically rotated out of the herd and replaced by younger female counterparts after several years of birthing terminal offspring (typically after birthing 2-8 offspring). The females that are rotated in are derived either from the female offspring of the herd, or imported from outside the herd. Larger cattle rearing operations that employ more than one herd have the ability to rotate into a herd a female or stud from another herd that has a defined familial relationship to the herd into which the female or stud is being rotated. This allows a producer of cattle to better predict and control the genetic makeup of the eventual offspring.

Cloning is a significant tool that can be employed by cattle producers to further improve the characteristics and value of offspring generated by their herd. As previously discussed herein, the ability to clone cattle using post-mortem tissue offers substantial breeding potentials. These benefits are even more pronounced in a controlled breeding situation wherein opportunities exist to:
revive the genetics of a bull that exhibits exceptional traits but whose genetic potential was previously cut off via castration or sacrifice,
integrate cloned bulls having known genetic traits into the breeding process, and
perpetuate the genetics of superior females.

Therefore, in one embodiment, the invention provides a method of producing cattle in a cattle breeding operation comprising (a) providing a herd comprising a plurality of female cattle and a male stud, (b) mating (via natural or artificial insemination, IVF, or otherwise) the female cattle and the male stud to produce a plurality of male and female offspring, (c) cloning a progenitor male or female offspring from the plurality of male and female offspring to produce a clone, (d) replacing the stud bull or one cow of said female cattle with said clone, and (e) repeating step (b) one or more times to produce a plurality of improved male and female offspring.

A cattle breeding operation is defined as one or more herds under solitary operational control, such that transfer of female and male cows among herds may occur as needed to carry out a defined program for narrowing and/or enhancing the genetic makeup of select herds or the entire operation. The composition of the herds is not static, and it will be understood that a step of the invention, when repeated, need not be performed precisely with the same herd composition or male stud as a previous step. Indeed, the elegance of the instant invention resides in the ability to continuously replace males or females in a herd with genetically superior clones whenever superior progenitor offspring are identified, and thereby to rapidly confine and enhance the genetic makeup of a particular herd. Thus, in another embodiment the invention further comprises:
a) cloning an improved progenitor male offspring from said improved male and female offspring to produce an improved male clone,
b) replacing the male stud with said improved male clone, and
c) repeating step (b) one or more times to produce a plurality of twice improved male and female offspring.

When the female genetics are cloned in successive generations the invention further comprises:
f) cloning an improved progenitor female offspring from said improved male and female offspring to produce an improved female clone;
g) replacing a female cow in said herd with said improved female clone; and
h) repeating step (b) one or more times to produce a plurality of twice-improved male and female offspring.

A number of variations of this general theme can be practiced to further enhance the utility of the instant invention. For example, the genetics can be propagated by introducing the clone to its parental herd or a different herd within the operation. Of course, when the clone is introduced to its parental herd care will usually be taken to avoid mating of the clone with its parent by removing the parent from the herd.

Similarly, the progenitor male or female offspring can be cloned one or more times. Cloning a progenitor male offspring more than once allows a narrowing of genetics within an entire breeding operation by associating the plurality of male clones with a corresponding number of herds. In contrast, cloning of a progenitor female offspring more than once allows one to introduce a plurality of genetically identical female clones to the same herd. To assure some genetic variability, a second progenitor female offspring can also be cloned more than once, and a second set of female clones can also be introduced to the herd. In this way, a herd may comprise 25, 15, 10, or 5 or less sets of female clones (each set being defined as derived from a unique progenitor female).

Nuclear Transfer

In a nuclear transfer procedure, a nuclear donor cell, or the nucleus thereof, is introduced into a recipient cell. Nuclear transfer procedures are known in the literature as described in Campbell, et al., “Theriogenology 43 181 (1995); Collas, et al., Mol. Reprod. Dev. 38 264-267 (1994); Keefer, et al., Biol. Reprod. 50 935-939 (1994); Sims, et al., Proc. Nat'l. Acad. Sci. USA 906143-6147 (1993); WO-A-9426884; WO-A-9424274; WO-A-9807841; WO-A-9003432; U.S. Pat. No. 4,994,384; and U.S. Pat. No. 5,057,420, each of which is incorporated herein by reference in its entirety.

Oocytes

The term “oocyte” is used to describe the mature animal ovum which is the final product of oogenesis and also the precursor forms being the oogonium, the primary oocyte and the secondary oocytc respectively. Unless otherwise specified herein, the term “oocyte” refers to an unfertilized egg in its natural nucleated state or its enucleated state (i.e., the genetic material that is typically present in the nucleus has been removed). The genetic material typically present in the oocyte nucleus is also referred to herein as maternal genetic material. Maternal genetic material does not include mitochondrial DNA. Unless otherwise specified herein, the term “(Kecyte” includes oocytes that are either activated or not activated. “Donor genetic material” is the genetic material, obtained from a donor cell, that is introduced into an oocyte. Donor genetic material contains the genetic material that is to be cloned and be present in the cloned non-human mammal. A nuclear transfer embryo or “NT embryo” is the result of introducing donor genetic material into an oocyte, and activating the embryo to induce mitogenesis. Thus, an NT embryo is the nuclear transfer equivalent of a fertilized egg. An NT embryo exists at such time whether or not the maternal genetic material is removed from the oocyte before transfer (i.e., the oocyte is enucleated). A one cell NT embryo is also referred to as a zygote. In some aspects of the present invention, nuclear transfer unit or “NT unit” is produced as a stage that precedes the NT embryo. An “NT unit” is the result of translocating the nuclear material from a donor cell into an oocyte, for instance into the perivitelline space (i.e., the space between an oocyte and the zona pellucida). An NT embryo may contain the maternal genetic material that was originally present in the oocyte.

Bovine and porcine oocytes are preferably from about 130 to about 230 microns in diameter when aspirated from the follicle. In more preferred embodiments, the oocytes are from about 150 to about 200 microns, and most preferably are about 180 microns in diameter.

Typically, oocytes are obtained from the ovaries or reproductive tract of a mammal. Slaughterhouse materials provide a readily available source of oocytes. Alternatively, oocytes can be surgically removed and used in the methods of the present invention. Methods for isolation of oocytes are well known in the art. For instance, the collection of immature bovine oocytes is described by Wells, et al. (Biol. Reprod., 60, 996-1005 (1999)), and collection of immature porcine oocytes is described by Abeydeera, et al. (Zygote 7, 203-10 (1999)) and Stice, et al., U.S. Pat. No. 5,945,577). Whole oocytes or bisected oocytes can be used in the present methods. Preferably whole oocytes are used.

Oocytes may be isolated from ovarian follicles at any stage of development, including primordial follicles, primary follicles, secondary follicles, growing follicles, vesicular follicles, maturing follicles, mature follicles, and graafian follicles. The selection of oocytes from porcine ovaries is carried out manually from follicles which are preferably at least about 2 mm in size, and more preferably about 3-8 mm in size. Materials and methods for isolating oocytes from various developmental stages of ovarian follicles are known to those skilled in the art. See, e.g., Laboratory Production of Cattle Embryos, 1994, Ian Gordon, CAB International; Anatomy and Physiology of Farm Animals (5th ed.), 1992, R. D. Frandson and T. L. Spurgeon, Lea & Febiger. In practice, a cumulus oocyte complex (COC) is aspirated from a follicle and the COC is subsequently matured in vitro. Alternatively, in vivo derived oocytes are stripped of their cumulus cells immediately after collection from the donor animals and used in the methods of the present invention. Methods for removing cumulus cells are known to the art (Tao, et al., Anim. Reprod. Sci., 56, 13341 (1999); Stice, et al., (U.S. Pat. No. 5,945,577). Prior to use, the stage of meiosis of the oocytes is determined using methods known to the art.

In vitro derived oocytes are initially collected from an animal, typically by aspiration of ovarian follicles, while the oocytes are immature. An immature oocyte is an oocyte that is in prophase. Typically, immature oocytes are subsequently cultured in media and allowed to mature under in vitro conditions. Media that can be used for the in vitro maturation of oocytes are referred to herein as maturation media or in vitro maturation (OM) medium. Examples include Tissue Culture Medium-199 (TCM-199), Waymouths, and NCSU-23 (described in Abeydeera, et al., (Zygote 7, 203-10 (1999)). Preferably TCM-199 is used for cows and NCSU-23 or TCM-199 is used for pigs. The in vitro maturation of oocytes is known to the art. (See, e.g., Prather, et al., Differentiation, 48: 1-8, 1991; Wang, et al., (J. Reprod. Fertil. 111 101-108(1997)).

A variety of other media well known to a person of ordinary skill in the art can be used for maturing oocytes in vitro. See, e.g., (i) Alm & Hinrichs, 1996, J. Reprod. Fert. 107: 215-220 and Alm & Torner, 1994, Theriogenology 42: 345-349 for equine oocytes; (ii) Ledda, et al., 1997, Journal of Reproduction and Fertility 109: 73-78; Byrd, et al., 1997, Theriogenology 47: 857-864; Wilmut, et al., 1997, Nature 385: 810-813; and LeGal, 1996, Theriogenology 45:1177-1 for caprine and ovine oocytes; (iii) Lorenzo, et al., 1996, Journal of Reproduction and Fertility 107: 109-117 and Jelinkova, et al., 1994, Molecular Reproduction and Development 37: 210-215 for leporidine oocytes; (iv) Nickson, et al., 1993, J. Reprod. Fert. (Suppl. 47): 231-240; Yamada, et al., 1993, J. Reprod. Fert. (Suppl. 47): 227-229; and Mahi & Yanagimachi, 1976, Journal of Experimental Zoology 196; 189-196 for canine oocytes; (v) Fukui et al., 1991, Theriogenology 35: 499-512 and Pollard, et al., 1995, Theriogenology 43: 301 for cervidine oocytes; and (vi) Del Campo, et al., 1995, Theriogenology 43:21-30; and Del Campo, et al., 1994, Theriogenology 41:187 for camelid oocytes. Oocytes may be cryopreserved and then thawed before placing the oocytes in maturation medium.

Typically, an oocyte is considered mature when it has reached metaphase II (MII) of the meiotic cell cycle. However, as explained by Stice, et al. in U.S. Application Pub. No. 2001/0053550, oocytes are also sufficiently mature at metaphase I (MI), and can also be used in the methods of the present invention. When used herein, unless otherwise expressly stated, the term “matured” oocyte refers to an oocyte that has reached mataphase II as determined by accepted morphological characteristics.

A recipient oocyte is preferably, but need not be, enucleated, when the nuclear transfer occurs. An oocyte can also be rendered “functionally enucleated,” for example by ultraviolet irradiation. See, e.g., Bradshaw, et al. (1995), Molecular Reproduction and Development 41: 505-12.

In vivo derived oocytes can be obtained from non-superovulated or superovulated donors. Donors can be induced to superovulate by methods known to the art. For instance, superovulated pig or cow donors can be obtained by treatment with PMSG (pregnant mare serum gonadotrophin) or FSH (follicle stimulating hormone). Preferably, oocytes are obtained from the donor animal when the donor is shortly (about 12 hours) after the onset of estrus. The period of time after the onset of estrus within which the oocytes can be obtained depends on the type of animal and is known to the art. For instance, if the donor animal is a cow or a pig the oocytes are preferably obtained within about 24 hours or about 48 hours of the onset of estrus, respectively.

Donor Cells

Donor genetic material contains the genetic material that is to be introduced into an oocyte and be present in the cloned non-human mammal. Donor genetic material can be isolated from a donor cell, i.e., the cell in which the genetic material is normally present. For instance, a nucleus or metaphase plate may be isolated from the donor cell and then introduced into an oocyte. A metaphase plate is described in further detail hereinbelow. Alternatively and preferably, the donor genetic material is not isolated from the donor cell before the donor genetic material is introduced into an oocyte, i.e., the donor cell itself is introduced into an oocyte, typically by introducing the donor cell into the perivitelline space of an oocyte and then fusing the donor cell with the oocyte as described hereinbelow. Optionally, donor genetic material includes DNA that is genetically engineered or transgenic.

The donor cells used in the methods of the present invention can be undifferentiated or differentiated cells, preferably differentiated. Differentiated mammalian cells are those cells which are beyond the early embryonic stage. More particularly, the differentiated cells are those from at least beyond the embryonic disc stage (for instance, about day 10 of bovine embryogenesis, or about day 8 of pig embryogenesis). Embryogenic stages from at least beyond the embryonic disc stage are referred to herein as late embryogenic stage. Fetal stage cells are those cells that are at least about day 20 to at least about day 30 of embryogenesis up to the time of birth. Adult stage cells are those present in an animal after birth. The differentiated cells may be derived from ectoderm, mesoderm or endoderm; preferably they are derived from mesoderm or endoderm.

Non-human mammalian cells for use as donor cells may be obtained by methods known to the art. Mammalian cells useful in the present invention include cells of the body, including, by way of example, epithelial cells, neural cells, epidermal cells, keratinocytes, hematopoietic cells, melanocytes, chondrocytes, lymphocytes (B and T lymphocytes), erythrocytes, macrophages, monocytes, mononuclear cells, fibroblasts, cardiac muscle cells, and other muscle cells. The mammalian cells that can be used in the methods of the present invention may be obtained from different organs, e.g., skin, lung, pancreas, liver, stomach, intestine, heart, reproductive organs, bladder, kidney, urethra and other urinary organs. The mammalian cells may be somatic or diploid germ cells obtained from embryo, fetus, or adult tissue, or from cultured cell lines, preferably adult tissue. The use of adult cells is advantageous as it allows the cloning of animals having desirable characteristics. These are just examples of suitable cells that can be used as a source of donor genetic material. Preferably, the cells are fibroblasts or granulosa cells.

In one aspect of the invention, the donor cell, whether it is introduced directly into an oocyte or used as a source of a donor nucleus or a donor metaphase plate that is introduced into an oocyte, is a quiescent cell (i.e., a cell at G0, see, for instance, Wilmut, et al., Nature, 385, 810-3 (1997); Campbell, et al., WO 97/07669), a proliferating cell (Stice, et al., U.S. Pat. No. 5,945,577), a metaphase cell, a cell arrested at metaphase, or a cell arrested at late G1 phase. Whether a donor cell is quiescent, proliferating, at metaphase, arrested at metaphase, or arrested at late G1 phase can be determined by methods known to the art. For example, a donor cell at metaphase is a cell that has progressed through the cell cycle including the prophase stage of mitosis; the centromeres joining the condensed sister chromatids are present in the region of the equatorial plane of the cell, and the nuclear membrane is absent. The appearance of the chromosomes of a metaphase cell is known to the art and is referred to as the metaphase plate. For example, a donor cell at late G1 is a cell that has intracellular concentrations of regulatory proteins, for instance, cyclin A and cyclin E, that are higher than in cells at other cell cycle phases. A donor cell arrested at metaphase or arrested at late G1 phase is unable to proceed beyond metaphase or G1 into anaphase or S phase, respectively, and is therefore no longer proliferating. Quiescent cells are not in any of the four phases of the cell cycle (i.e., G1, S, G2, or M). Quiescent cells are typically considered as being in the G0 state so as to indicate that they would not normally progress through the cycle. The nucleus of a quiescent G0 cell is diploid. Thus, in contrast to a quiescent cell, a cell arrested at metaphase does not have a nucleus, and the DNA content is tetraploid. In contrast to a quiescent cell, a cell arrested at late G1 is prepared to undergo DNA replication but is still diploid.

Preferably, a donor cell is quiescent, arrested at G1, at metaphase, or arrested at metaphase. Placing the metaphase donor genetic material into an oocyte is advantageous because it facilitates additional exposure to cytoplasmic reprogramming factors needed for reprogramming donor genetic material that has been introduced into the oocyte. Placing the donor genetic material arrested at late G1 into an oocyte is advantageous because the donor nucleus is prepared to undergo DNA replication during S phase of the first cell cycle of the NT embryo.

Donor cells can be arrested in metaphase by exposing the cells to at least one arresting agent. Useful arresting agents include nocodazole, demicolchin, colchicine, colcemid, paclitaxel, docetaxel, otoposide, vinblastine, vincristine, vinorelbine, monastrol, and taxol, preferably nocodazole. Preferably, the arrested state of the donor cell is reversible, i.e., the cell resumes proliferating when the arresting agent(s) is removed. The exposure of a population of donor cells to an arresting agent typically does not result in arrest of all the donor cells, thus those cells that are arrested (and therefore typically at metaphase) can be separated from those that are not arrested. Cells arrested at metaphase typically have an altered morphology that allows arrested cells to be separated. For instance, arrested cells grown on a surface and then exposed to an arresting agent have a “rounded up” appearance while proliferating cells are relatively flat.

Donor cells may be arrested at G1 by exposing the cells to at least one arresting agent. Useful arresting agents include mimosine, aphidocoline, and inhibitors of CDK2 kinase, including for instance roscovitine or olomoucine (see, for instance, Alessi, et al., Exp. Cell Res., 245, 8-18 (1998)). Preferably, roscovitine or olomoucine, more preferably roscovitine, are used, although contact inhibition is another particularly preferred technique. Preferably, the arrested state of the donor cell is reversible, i.e., the cell resumes proliferating when the arresting agent(s) is removed. The exposure of a population of donor cells to an arresting agent typically does not result in arrest of all the donor cells, thus those cells that are arrested (and therefore typically at late G1) can be separated from those that are not arrested. Cells at G1 typically have an altered morphology that allows arrested cells to be separated. For instance, arrested cells are typically smaller in size than those cells that are not arrested at late G1. Preferably, donors cells arrested in late G1 having a size of about 15 μM to about 20 μM in size are selected for introduction into an oocyte. Donor cells can be induced to enter quiescence by employing various conventional methods of inducing quiescence such as serum starvation and contact inhibition (i.e. growth in culture to confluence). A preferred method is contact inhibition.

Donor genetic material can be isolated from quiescent cells, proliferating cells, cells that are at metaphase, cells that are arrested at metaphase, or cells arrested at late G1 using methods known to the art (see, for instance, Collas and Barnes, Mol. Reprod. Dev., 38, 264-267 (1994)). Typically, a donor nucleus can be isolated by removing the cell membrane, or further isolated by removing at least some of the cytoplasm that normally surrounds the donor nucleus.

A variety of methods for culturing donor cells exist in the art. See, e.g., Culture of Animal Cells: a manual of basic techniques (3rd edition), 1994, Freslney (ed.), Wiley-Liss, Inc.; Cells: a laboratory manual (vol. 1), (1998), Spector, Goldman, Leinwand (eds.), Cold Spring Harbor Laboratory Press; and Animal Cells: culture and media, 1994, Darling & Morgan, John Wiley and Sons, Ltd.

Nuclear Translocation

A nuclear donor can be translocated into an oocyte, most preferably an enucleated oocyte, using a variety of materials and methods that are well known to a person of ordinary skill in the art. Isolated donor genetic material may be injected directly into an oocyte to produce the NT embryo (see, for instance, Collas and Barnes, Mol. Reprod. Dev., 38, 264-267 (1994); and Tao, et al., Anim. Reprod. Sci., 56, 13341 (1999)). A peizo element based micromanipulator may be used to facilitate microinjection tasks (see, for instance, Wakayama, et al., Nature, 394, 369-74 (1998)). It is expected that a nuclear membrane will form around a metaphase plate that is introduced into an oocyte.

Alternatively, a single donor cell of the same species as the oocyte may be introduced by fusing the cell with the oocyte after the donor cell is placed in the perivitelline space of the oocyte (i.e., the space between an oocyte and the zona pellucida) to produce an NT unit Such methods are known to the art (see, for instance, Stice, et al., (U.S. Pat. No. 5,945,577)). A variety of electrofusion media can be used including e.g., sucrose, mannitol, sorbitol and phosphate buffered solution. Fusion can also be accomplished using Sendai virus as a fusogenic agent (Graham, Wister Inot. Symp. Monogr., 9, 19, 1969), or by using polyethylene glycol (PEG) (Susko-Parrish, et al., U.S. Pat. No. 5,496,720). Other examples of non-electrical means of cell fusion involve incubating in solutions comprising trypsin, dimethylsulfoxide (DMSO), lectins, and agglutinin viruses. Fusion of the donor cell and the oocyte that make up an NT unit result in an NT embryo.

Typically, in electrofusion of porcine oocytes and donor cells, a fusion pulse ranging from about 150 V/mm to about 350 V/mm, more preferably about 250 V/mm, is used. The duration of the pulse may be about 20 μseconds. For electrofusion of bovine oocytes and donor cells, a fusion pulse of about 40 V/150 μm may be used. The duration of the pulse is about 20 μseconds. Multiple pulses can also be used successfully to induce cell fusion. The result is a one-cell NT embryo.

NT Embryo

If desired, an NT embryo can be cultured in media. The type of media can depend on the species of oocyte. For instance, for pig cells, NCSU-23 or other pig embryo culture medium (see, for instance, Tao, et al., Anim. Reprod. Sci., 56, 13341 (1999)) can be used. Preferably, for pig cells, a sequential media system is used. The first medium of the sequential media system is a bicarbonate-buffered culture medium that includes alanine, alanyl-glutamine, asparagine, aspartic acid, calcium chloride, EDTA, glucose, glutamate, glycine, human serum albumin, magnesium sulphate, penicillin G, potassium chloride, proline, serine, sodium bicarbonate, sodium chloride, sodium hydrogen phosphate, sodium lactate, sodium pyruvate, and taurine is used. Such a culture medium is available under the trade designation G1.2(Vitrolife, Inc., Englewood Colo.). The second medium of the sequential media system is a bicarbonate-buffered culture medium that includes alanine, alanyl-glutamine arginine, asparagine, aspartic acid, calcium chloride, calium pantothenate, choline chloride, cystine, folic acid, glucose, glutamate, glycine, Histidine, human serum albumin, i-inositol, isoleucine, leucine, lysine, magnesium sulphate, methionine, niacinamide, penicillin G, phenylalanine, potassium chloride, proline, pyridoxine, riboflavin, serine, sodium bicarbonate, sodium chloride, sodium Hydrogen phosphate, sodium lactate, sodium pyruvate, thiamine, threonine, tryptophan, tyrosine, valine. Such a culture medium is available under the trade designation G2.2 (Vitrolife, Inc.). This sequential media system is referred to herein as G1/G2, or G1.2/G2.2. For cow cells, G1/G2, KSOM, CR, or TCM-199, G1/G2, can be used. The NT embryo is typically incubated for up to about 10 hours. Preferably, an NT embryo is not incubated so long that the chromosomes begin to disassociate from each other, and/or micronuclei are formed after activation. Alternatively, an NT embryo need not be cultured in media.

If the oocyte used to produce the NT embryo was not enucleated, the NT embryo, whether incubated in medium or not, can optionally be enucleated. Enucleation of an NT embryo involves removal of maternal genetic material from the NT embryo, but not removal of donor genetic material. Enucleation of an NT embryo is discussed hereinbelow. Preferably, when the oocyte used to produce the NT embryo was not enucleated, the method of the invention preferably includes enucleation of the NT embryo. Further, if the oocyte used to produce the NT embryo was not activated, the method preferably includes activation of the NT embryo. Activation of an NT embryo can be performed either before or after the enucleation step.

Enucleation

Oocytes may be enucleated before introduction of donor genetic material. Enucleation of oocytes may be accomplished microsurgically using a micropipette to remove the polar body and the adjacent cytoplasm, or by chemical treatment (see, for instance, Baguisi, et al., Theriol., 53, 290 (2000)). If enucleation is performed prior to introduction of donor genetic material, it may be conducted using methods previously described for enucleating MII oocytes (Tao, et al., Anim. Reprod. Sci., 56, 133-41 (1999)) or by methods such as described by Goto, et al., (Anim. Sci. J. 70, 243-245 (1999)). The oocytes may then be screened to identify those successfully enucleated. This screening can be done by staining the oocytes with a detectable marker that specifically binds to DNA (for instance, 1 μg/ml 33342 Hoechst dye in BEPES buffered hamster embryo culture medium (HECM, Seshagine, et al., Biol. Reprod., 40, 544-606, (1989)), and then viewing under ultraviolet irradiation for less than 10 seconds either the oocytes or the cytoplasm and maternal genetic material removed during the enucleation procedure. The oocytes that have been successfully enucleated can then be placed in a suitable culture medium, e.g., TCM-199, G1/G2, or CR1aa plus 10% serum (Stice, et al., U.S. Pat. No. 5,945,577).

In vitro matured oocytes enucleated before introduction of donor genetic material can be enucleated when they are at the appropriate stage, e.g., immature germinal vesicle, maturing (MI to MII), or mature. In vivo matured oocytes enucleated before introduction of donor genetic material can be enucleated after isolation, preferably immediately after isolation.

If the oocyte used to produce the NT embryo was not enucleated, then the NT embryo can be enucleated. Within the NT embryo, the maternal genetic material can be distinguished from the donor genetic material by, for instance, the position of the donor nucleus within the NT embryo, formation of the first polar body, or a combination thereof. The known location of the donor genetic material within the NT embryo is based on where it was placed in the perivitelline space in relation to the location of the maternal genetic material. The maternal genetic material is near the opening placed in the zona pellucida during transfer of the donor genetic material, preferably the donor genetic material is placed away from that area. Therefore that area of cytoplasm (near the opening in the zona) can be removed via either enucleation pipette or by expulsion of cytoplasm through the opening in the zona, preferably by enucleation pipette (see, e.g., Prather, et al., Biol. Rerod., 37, 859 (1987); and Goto, et al., Anim. Sci. J, 20, 243-245 (1999)). When MI oocytes are employed in the nuclear transfer process, the oocyte may progress in meiosis to MII after introduction of the donor genetic material. If so, then the first polar body can also be used as landmark to find the maternal genetic material. Hoechst dye can be used to visualize genetic material, including confirming the presence of the maternal genetic material in the removed cytoplasm. These methods may be used alone or in conjunction with each other to verify location of chromosomes and verify enucleation of the oocyte.

An NT embryo that contains both maternal and donor genetic material need not be immediately enucleated or, in some aspects of the invention, is not enucleated at all. That is, the NT embryo will at least transiently contain both maternal genetic material and donor genetic material. For instance, Willadsen, et al. (Nature, 320, 63-65 (1986)), used non-enucleated NT embryos derived from MII oocytes to produce cloned sheep embryos. It is expected that maternal genetic material may contribute to only the placenta, thus the cells that develop to eventually form a fetus or offspring would not contain maternal genetic material.

Activation

An oocyte or an NT embryo may be activated using artificial activation methods known to the art (see, for instance, Susko-Parrish, et al., (U.S. Pat. No. 5,496,720); and Stice, et al., (U.S. Pat. No. 5,945,577)). An oocyte may be activated before introduction of donor genetic material, or at the same time as the introduction of donor genetic material. Alternatively and preferably, an NT embryo may be activated. Typically, when an oocyte is activated before introduction of donor genetic material, the activated oocyte is used immediately or within about 10 hours after activation. When an NT embryo is activated, activation is done at about the same time as introduction of the donor genetic material or up to about 10 hours following introduction.

Activation may include the use of agents that decrease protein phosphorylation in the cell, decrease protein synthesis by the cell, or increase the level of cations in the cell. Protein phosphorylation can be decreased by the use of agents that inhibit phosphorylation, including, for instance, a serine-threonine kinase inhibitor like 6-dimethylaminopurine, staurosporine, 2-aminopurine, or sphingosine. Protein phosphorylation can also be decreased by the use of agents that cause dephosphorylation of proteins, including for instance phosphatases A or B. Agents that decrease protein synthesis by the cell include, for instance, cycloheximide. Agents that increase the level of cations in the cell include, for instance, ionomycin, ionophores, ethanol, media free of Mg⁺⁺ and Ca⁺⁺, phorbol esters, and electrical shock. Other agents that can be used include thimerasol and DTT (Machaty, et al., Biol. Reprod., 57, 1123 (1997)).

Specific examples of activation methods are listed below.

1. Activation by Ionomycin and DMAP: 1—Place oocytes in lonomycin (5 μM) with 2 mM of DMAP for 4 minutes; 2—Move the oocytes into culture media with 2 mM of DMAP for 4 hours; 3—Rinse four times and place in culture.

2. Activation by Ionomycin, DMAP and Roscovitin: 1—Place oocytes in Ionomycin (5 μM with 2 mM of DMAP for four minutes; 2—Move the oocytes into culture media with 2 mM of DMAP and 200 microM of Roscovitin for three hours; 3—Rinse four times and place in culture.

3. Activation by exposure to Ionomycin followed by cytochalasin and cycloheximide: 1—Place oocytes in Ionomycin (5 microM) for four minutes; 2—Move oocytes to culture media containing 5 μg/ml of cytochalasin B and 5 μg/ml of cycloheximide for five hours; 3—Rinse four times and place in culture.

4. Activation by electrical pulses: 1—Place eggs in mannitol media containing 100 μM CaCl₂; 2—Deliver three pulses of 1.0 kVcm⁻¹ for 20 μsec, each pulse 22 minutes apart; 3—Move oocytes to culture media containing 5 μg/ml of cytochalasin B for three hours.

5. Activation by exposure with ethanol followed by cytochalasin and cycloheximide: 1—Place oocytes in 7% ethanol for one minute; 2—Move oocytes to culture media containing 5 μg/ml of cytochalasin B and 5 μg/ml of cycloheximide for five hours; 3—Rinse four times and place in culture.

6. Activation by microinjection of adenophostine: 1—Inject oocytes with 10 to 12 picoliters of a solution containing 10 μM of adenophostine; 2—Put oocytes in culture.

7. Activation by microinjection of sperm factor: 1—Inject oocytes with 10 to 12 picoliters of sperm factor isolated, e.g., from primates, pigs, bovine, sheep, goats, horses, mice, rats, rabbits or hamsters; 2—Put eggs in culture.

8. Activation by microinjection of recombinant sperm factor.

9. Activation by Exposure to DMAP followed by Cycloheximide and Cytochalasin B: 1—Place oocytes or NT units in about 2 mM DMAP for about one hour, followed by incubation for about two to twelve hours, preferably about eight hours, in 5 μg/ml of cytochalasin B and 20 μg/ml cycloheximide.

Activation of porcine oocytes and NT embryos may use about 1% to about 20% ETOH, preferably 8% ETOH in KSOM or G1/G2 culture medium for 10 minutes followed by about 1 mM to about 10 mM DMAP, preferably about 2 mM DMAP in KSOM or G1/G2 for 5 hours. Preferably, porcine oocytes and NT embryos are activated by applying two pulses of from about 50 V/mm to about 200 V/mm (direct current), more preferably about 75 V/mm. The two pulses are each preferably about 60 pseconds long, and preferably separated by about a 5 second interval. Preferably, the activation is done in Zimmerman fusion media (Zimmerman, et al., Membrane Biol., 67, 165-182 (1982)).

Bovine oocytes and NT embryos may be activated by the method of Yang, et al. (Biol. Reprod., 42(Suppl 1), 117 (1992)), more preferably, by exposing bovine oocytes to about 1 M to about 100 μM ionomycin, preferably about 50 μM ionomycin, for 10 minutes and about 1 μg/ml to about 100 μg/ml cycloheximide, preferably about 10 μg/ml cycloheximide, for about 2 hours to about 10 hours, preferably about 6 hours. Preferably, bovine oocytes and NT embryos are activated by exposure to agents that increase the level of cations in the cell, followed by exposure to agents that decrease protein synthesis in the cell and/or agents that are microfilament inhibitors. Most preferably, bovine oocytes and NT embryos are exposed to about 1 μM to about 100 μM calcium ionophore, preferably about 5 μM calcium ionophore, for about 10 minutes. This is followed by incubation in about 1 μg/ml to about 10 μg/ml cytochalasin B, preferably about 5 μg/ml cytochalasin B, and about 1 μg/ml to about 100 μg/ml cycloheximide, preferably about 10 μg/ml cycloheximide, for about 1 hour. This is followed by incubation in about 1 μg/ml to about 10 μg/ml cycloheximide, preferably about 10 μg/ml cycloheximide, for about 5 hours. Preferably, after the activation treatments, bovine NT embryos are cultured in BARC medium (Powell, et al., Theriogen., 55, 287 (2001)).

Whether a porcine or bovine oocyte or a porcine or bovine NT embryo has been activated can be determined by observing swelling of the donor nucleus, and cleavage of the embryo about 10 hours to about 30 hours after activation.

Instead of using artificial activation methods, or in conjunction with artificial activation methods, fertilized oocyte cytoplasm can be used to activate an oocyte or an NT embryo. The use of fertilized oocyte cytoplasm to activate an oocyte or an NT embryo is referred to herein as “natural activation.” Fertilized oocyte cytoplasm can be obtained by removal of cytoplasm from an oocyte that has been fertilized by a sperm. Fertilized oocyte cytoplasm can be removed by pipette and then injected directly into the oocyte or NT embryo that is to be activated. It is expected that fertilized oocyte cytoplasm can be injected in volumes up to between about 10% and about 50% the volume of the oocyte or NT embryo that is to be activated.

As noted, activation may be effected before, simultaneous, or after nuclear transfer. In general, activation will be effected about 40 hours prior to nuclear transfer and fusion to about 40 hours after nuclear transfer and fusion, more preferably about 24 hours before to about 24 hours after nuclear transfer and fusion, and most preferably from about 4 to 9 hours before nuclear transfer and fusion to about 4 to 9 hours after nuclear transfer and fusion. Activation is preferably effected after or proximate to in vitro maturation of the oocyte.

Assessment of Successful Nuclear Reprogramming and Transfer of Activated NT Embryos

Successful nuclear reprogramming is evaluated by determining if activated NT embryos develop to the blastocyst stage. For both pig and cow, development of an activated NT embryo to blastocyst is typically complete in seven days, and typically includes the trophoblast and inner cell mass.

An activated NT embryo may be transferred immediately into a recipient animal or cultured for up to about 8 days in, for instance, KSOM medium, NCSU-23 medium, BARC medium, G1.2/G2.2 culture medium, or others well known to the art (see for instance Stice, et al., U.S. Pat. No. 5,945,577; Wells, et al., Biol. Reprod., 60, 996-1005 (1999); and Tao, et al., Anim. Reprod. Sci., 56, 133-41 (1999)). Preferably, an activated NT embryo is cultured for between about 12 hours to about 36 hours (for porcine NT embryos) or for about 7 to about 8 days (for bovine NT embryos). Then, intact NT embryos (some cleaved) are transferred into a synchronous recipient animal, i.e., the transferred NT embryo is at the same stage, or about a day before or a day after, as a fertilized embryo would be in the recipient. For pigs, from about one to about 300 NT embryos can be transferred into each recipient female but typically about 50 to about 150 embryos are transferred and ideally 100 embryos are transferred. Methods of surgical and non-surgical transfer in animals is well known in the art. For instance, surgical and non-surgical transfer in pigs is described by Cumock, et al., (Amer. J. Vet. Res., 37, 97-98 (1976)), and Hazeleger, et al., (Theriogenol., 51, 81-91 (1999)). Preferably, the animal is of the same species as the donor genetic material of the NT embryo.

Ultrasound and non-return to estrus is used to determine which recipients are pregnant. If needed for tissue or cell transplantation NT fetuses can be harvested during the pregnancy through surgical recovery. If live calves or pigs are desired the pregnancy lasts approximately 285 days or 114 days respectively, and some offspring may require neonatal assistance in the form of oxygen supplementation and other interventions (Hill, et al., Theriogenol., 51, 1451 (1999)).

Other Sources of Information

Where descriptions of oocyte maturation, oocyte enucleation, cell activation, in vitro embryo development, and other processes are described herein in relation to mammals in general, the following references provide additional descriptions of such process for specific mammals. The following references are provided to aid the reader in understanding the invention and are not admitted to describe or constitute prior art to the present invention. With regard to suids, researchers have reported materials and methods for oocyte maturation, oocyte enucleation, cell activation, in vitro embryo development, and other processes. See, e.g., Grocholová, et al., 1997, J. Exp. Zoology 277: 49-56; Schoenbeck, et al., 1993, Theriogenology 40: 257-266; Prather, et al., 1989, Biology of Reproduction 41: 414-418; Prather, et al., 1991, Molecular Reproduction and Development 28: 405-409; Jolliff & Prather, 1997, Biol. Reprod. 56:544-548; Mattioli, et al., 1991, Molecular Reproduction and Development 30: 109-125; Terlouw, et al., 1992, Theriogenology 37: 309; Prochazka, et al., 1992, J. Reprod. Fert. 96: 725-734; Funahashi, et al., 1993, Molecular Reproduction and Development 36: 361-367; Prather, et al., Bio. Rep. Vol. 50 Sup 1: 282; Nussbaum, et al., 1995, Molecular Reproduction and Development 41: 70-75; Funahashi, et al., 1995, Zygote 3: 273-281; Wang, et al., 1997, Biology of Reproduction 56: 1376-1382; Piedrahita, et al., 1989, Biology of Reproduction 58: 1321-1329; Machaty, et al., 1997, Biology of Reproduction 57: 85-91; and Machaty, et al., 1995, Biology of Reproduction 52: 753-758.

With regard to bovids, researchers have reported materials and methods for oocyte maturation, oocyte enucleation, cell activation, in vitro embryo development, and other processes. See, e.g., U.S. Pat. Nos. 5,453,357 and 5,670,372, entitled “Pluripotent Embryonic Stem Cells and Methods of Making Same,” Hogan; Sims & First, 1993, Theriogenology 39:313; Keefer, et al., 1994, Mol. Reprod. Dev. 38: 264-268; U.S. Pat. No. 4,994,384, “Multiplying Bovine Embryos,” Prather, et al.; U.S. Pat. No. 5,057,420, “Bovine Nuclear Transplantation,” Massey & Willadsen; Delhaise, et al., 1995, Reprod. Fert. Develop. 7: 1217-1219; Lavoir 1994, J. Reprod. Dev. 37: 413-424; PCT application WO 95/10599 entitled “Embryonic Stem Cell-Like Cells”; Stice, et al., 1996, Biol. Reprod. 54: 100-110; Strelchenko, 1996, Theriogenology 45: 130-141; WO 97/37009, entitled “Cultured Inner Cell Mass Cell-Lines Derived from Ungulate Embryos,” Stice and Golueke, published Oct. 9, 1997; U.S. Pat. No. 5,213,979, entitled “In vitro Culture of Bovine Embryos,” First, et al., May 25, 1993; U.S. Pat. No. 5,096,822, entitled “Bovine Embryo Medium,” Rosenkrans, Jr., et al., Mar. 17, 1992; Seidel and Elsden, 1997, Embryo Transfer in Dairy Cattle, W. D. Hoard & Sons, Co., Hoards Dairyman; Stice & Keefer, 1993, “Multiple generational bovine embryo cloning,” Biology of Reproduction 48: 715-719; Wagoner, et al., 1996, “Functional enucleation of bovine oocytes: effects of centrifugation and ultraviolet light,” Theriogenology 46: 279-284; Pieterse, et al., 1988, “Aspiration of bovine oocytes during transvaginal ultrasound scanning of the ovaries,” Theriogenology 30: 751-762; Saito, et al., 1992, Roux's Arch. Dev. Biol. 201: 134-141; and U.S. Pat. No. 5,496,720, entitled “Parthenogonic Oocyte Activation,” Mar. 5, 1996, Susko-Parrish, et al.

With regard to ovids and caprids, researchers have reported materials and methods for oocyte maturation, oocyte enucleation, cell activation, in vitro embryo development, and other processes. See, e.g., Willadsen, 1986, Nature 320: 63-66; Ruffing, et al., 1993, Biology of Reproduction 48: 889-904; Smith & Wilmut, 1989, Biology of Reproduction 40: 1027-1035; McLaughlin, et al., 1991, Theriogenology 35: 240; Campbell, et al., 1995, Theriogenology 43:181; Campbell, et al., 1996, Theriogenology 45: 286; Campbell, et al., 1996, Nature 380: (4-66; Wilmut, et al., 1997, Nature 385: 810-813; Ledda, et al., 1997, Journal of Reproduction and Fertility 109:73-78; Byrd, et al., 1997, Theriogenology 47: 857-864; Wilmut, et al., 1997, Nature 385: 810-813; LeGal, 1996, Theriogenology 45: 1177-1; Pawshe, et al., 1996, Theriogenology 46: 971-982; Gall, et al., 1993, Molecular Reproduction and Development 36: 500-506, Walker, et al., 1996, Biology of Reproduction 55: 703-708; and Gardner, et al. 1994. Biology of Reproduction 50: 390-400.

Transgenic Applications

In particularly preferred embodiments, embryos, fetuses and/or animals of the invention are transgenic. The term “transgenic” as used herein refers to an embryo, fetus or animal comprising one or more cells whose genome has been altered using recombinant DNA techniques. In preferred embodiments, a transgenic embryo, fetus, or animal comprises one or more transgenic cells. While germ line transmission is not a requirement of transgenic embryos, fetuses, or animals as that term is used herein, in particularly preferred embodiments a transgenic embryo, fetus, or animal can pass its transgenic characteristic(s) through the germ line. In certain embodiments, a transgenic embryo, fetus or animal expresses one or more exogenous genes, as exogenous RNA and protein molecules. Most preferably, a transgenic embryo, fetus or animal results from a nuclear transfer procedure using a transgenic nuclear donor cell.

Materials and methods readily available to a person of ordinary skill in the art can be utilized to convert the nuclear donor cells of the invention into transgenic cells. Once nuclear DNA is modified in a nuclear donor cell, embryos, fetuses, and animals arising from these cells can also comprise the modified nuclear DNA. Hence, materials and methods readily available to a person of ordinary skill in the art can be applied to nuclear donor cells to produce transgenic cloned and chimeric animals. See, e.g., EPO 254 166, entitled “Transgenic Animals Secreting Desired Proteins Into Milk;” WO 94/19935, entitled “Isolation of Components of Interest From Milk;” WO 93/22432, entitled “Method for Identifying Transgenic Pre-implantation Embryos;” WO 95/17085, entitled “Transgenic Production of Antibodies in Milk;” Hammer, et al., 1985, Nature 315: 580-685; Miller, et al., 1986, J. Endocrinology 120: 481488; Williams, et al., 1992, J. Ani. Sci. 70: 2207-2111; Piedrahita, et al., 1998, Biol. Reprod. 58: 1321-1329; Piedrahita, et al., 1997, J. Reprod. Fert. (suppl.) 52: 245-254; and Nottle, et al, 1997, J. Reprod. Fert. (suppl.) 52: 245-254, each of which is incorporated herein by reference in its entirety including all figures, drawings and tables.

Methods for generating transgenic cells typically include (1) assembling a suitable DNA construct useful for inserting a specific DNA sequence into nuclear: DNA of a cell; (2) transfecting the DNA sequence into cells; (3) allowing random insertion and/or homologous recombination to occur. A modification resulting from such a process may include insertion of a suitable DNA construct(s) into a target genome; deletion of DNA from a target genome; and/or mutation of a target genome.

DNA constructs can comprise a gene of interest as well as a variety of elements including regulatory promoters, insulators, enhancers, and repressors as well as elements for ribosomal binding to RNA transcribed from a DNA construct. DNA constructs can also encode ribozymes and anti-sense DNA and/or RNA. Moreover, DNA constructs can comprise a selection element, such as a gene for drug selection of transformants. These examples are well known to a person of ordinary skill in the art and are not meant to be limiting.

Due to effective recombinant DNA techniques available in conjunction with DNA sequences for regulatory elements and genes readily available in data bases and the commercial sector, a person of ordinary skill in the art can readily generate a DNA construct appropriate for establishing transgenic cells using materials and methods described herein. For example, transfection techniques are well known to a person of ordinary skill in the art and materials and methods for carrying out transfection of DNA constructs into cells are commercially available. For example, materials that can be used to transfect cells with DNA constructs are lipophillic compounds, such as Lipofectin™, Superfect™, LipoTAXI™, and CLONfectin™. Particular lipophillic compounds can be induced to form liposomes for mediating transfection of the DNA construct into the cells. In addition, cationic based transfection agents that are known in the art can be utilized to transfect cells with nucleic acid molecules (e.g., calcium phosphate precipitation, DEAE-dextran, polybrene, polyamine). Other techniques are known in the art that use protein-based or amphipathic polyamines as transfection reagents. Also, electroportation techniques known in the art can be utilized to translocate nucleic acid molecules into cells. Particle bombardment techniques are also known in the art for introducing exogenous DNA into cells.

Target sequences from a DNA construct can be inserted into specific regions of nuclear DNA by rational design of a DNA construct. Such design techniques and methods are well known to a person of ordinary skill in the art. See, U.S. Pat. No. 5,633,067, “Method of Producing a Transgenic Bovine or Transgenic Bovine Embryo;” DeBoer, et al., issued May 27, 1997; U.S. Pat. No. 5,512,205, “Homologous Recombination in Mammalian Cells;” Kay et al., issued Mar. 18, 1997; and PCT publication WO 93/22432, Method for Identifying Transgenic Pre-Implantation Embryos,” each of which is incorporated herein by reference in its entirety, including all figures, drawings, and tables. Once a desired DNA sequence is inserted into the nuclear DNA of a cell, the location of an insertion region as well as the frequency with which the desired DNA sequence has been inserted into the nuclear genome can be identified by methods well known to those skilled in the art.

Desired DNA sequences can be inserted into nuclear DNA of a cell to enhance the resistance of a cloned transgenic animal to particular parasites, diseases, and infectious agents. Examples of parasites include worms, flies, ticks, and fleas. A transgene can confer resistance to a particular parasite or disease by completely abrogating or partially alleviating symptoms of the disease or parasitic condition or by producing a protein which controls the parasite or disease. Examples of infectious agents include bacteria, fungi, and viruses. Examples of diseases include Atrophic rhinitis, Cholera, Leptospirosis, Pseudorabies, Pasturellosis, and Brucellosis. These examples are not limiting and the invention relates to any disease or parasite or infectious agent known in the art. See, e.g., Hagan & Brunets Infectious Diseases of Domestic Animals (7th edition), Gillespie & Tirnoney, 1981, Cornell University Press, Ithaca N.Y.

A wide variety of transcriptional and translational regulatory sequences may be inserted into nuclear DNA of a nuclear donor cell. Transcriptional and translational regulatory signals may be derived from viral sources, such as adenovirus, bovine papilloma virus, cytomegalovirus, simian virus or the like, whereas the regulatory signals can be associated with a particular gene sequence having a potential for high levels of expression. Additionally, promoters from mammalian expression products, such as actin, casein alpha-lactalbumin, uroplakin, collagen, myosin, and the like, may be employed. Transcriptional regulatory signals may be selected which allow for repression or activation, so that expression of a gene product can be modulated. Of interest are regulatory signals which can be repressed or initiated by external factors such as chemicals or drugs. These examples are not limiting and the invention relates to any regulatory elements. Other examples of regulatory elements are described herein.

A variety of proteins and polypeptides can be encoded by a gene harbored within a DNA construct suitable for creating transgenic cells. Those proteins or polypeptides include hormones, growth factors, enzymes, clotting factors, apolipoproteins, receptors, drugs, pharmaceuticals, bioceuticals, nutraceuticals, oncogenes, tumor antigens, tumor suppressors, cytokines, viral antigens, parasitic antigens, bacterial antigens and chemically synthesized polymers and polymers biosynthesized and/or modified by chemical, cellular and/or enzymatic processes. Specific examples of these compounds include proinsulin, insulin, growth hormone, androgen receptors, insulin-like growth factor I, insulin-like growth factor II, insulin growth factor binding proteins, epidermal growth factor, TGF-α, TGF-β, dermal growth factor, platelet derived growth factor (PDGF), angiogenesis factors (e.g., acidic fibroblast growth factor, basic fibroblast growth factor, and angiogenim), angiogenesis inhibitors (e.g., endostatin and angiostatin), matrix proteins (Type IV collagen, Type VII collagen, laminin), oncogenes (ras, fos, myc, erb, src, sis, jun), E6 or E7 transforming sequence, p53 protein, cytokine receptor, IL-1, IL-6, IL-8, IL-2, α, β, or γ IFN, GMCSF, GCSF, viral capsid protein, and proteins from viral, bacterial and parasitic organisms. Other specific proteins or polypeptides which can be expressed include: phenylalanine hydroxylase, α-1-antitrypsin, cholesterol-7β-hydroxylase, truncated apolipoprotein B, lipoprotein lipase, apolipoprotein E, apolipoprotein A1, LDL receptor, scavenger receptor for oxidized lipoproteins, molecular variants of each, VEGF, and combinations thereof. Other examples are antibodies (monoclonal or polyclonal), antibody fragments, clotting factors, apolipoproteins, drugs, tumor antigens, viral antigens, parasitic antigens, monoclonal antibodies, and bacterial antigens. One skilled in the art readily appreciates that these proteins belong to a wide variety of classes of proteins, and that other proteins within these classes or outside of these classes can also be used. These are only examples and are not meant to be limiting in any way.

A pig prepared by a method in accordance with any aspect of the present invention may be used as a source of tissue for transplantation therapy. Similarly, a pig embryo prepared in this manner or a cell line developed therefrom may also be used in cell-transplantation therapy. Accordingly, there is provided in a further aspect of the invention a method of therapy comprising the administration of porcine cells to a patient, wherein the cells have been prepared from an embryo or animal prepared by a method as described above. This aspect of the invention extends to the use of such cells in medicine, e.g. cell-transplantation therapy, and also to the use of cells derived from such embryos in the preparation of a cell or tissue graft for transplantation. The cells may be organized into tissues, for example, heart, lung, liver, kidney, pancreas, corneas, nervous (e.g. brain, central nervous system, spinal cord), skin, or the cells may be blood cells (e.g. haemocytes, i.e. red blood cells, leucocytes) or haematopoietic stem cells or other stem cells (e.g. bone marrow). A method of the present invention will therefore also find utility in the preparation of xenografts. These methods might include in vitro differentiation of embryonic cells for therapeutic transplantation into a patient, including situations where the cells are genetically modified to correct a medical defect. Such applications might include treatment of diseases such as diabetes, Parkinson's disease, motor neurone disease, multiple sclerosis, AIDS etc, or disease conditions characterized by a loss of function in the cells or an organ of an affected individual.

Because of the human antibody induced hyperacute rejection of natural porcine tissues, various strategies are employed to modify the tissue to avoid transplant rejection. In one particular embodiment, the −1,3-galactosyltransferase porcine gene in pigs is knocked out (i.e. the expression of the gene is suppressed) to minimize the risk or incidence of hyperacute rejection. Knock out methods are known in the art, and are described in detail in PCT publication no. WO 98/57538 of Machaty, et al.

Stem Cell Applications

The recipient cell into which the donor nucleus has been transferred may be cultured in vitro or in vivo until a suitable stage in embryonic development is reached. The invention includes the derivation of a cell line from desired cells of the embryo, e.g. inner cell mass cells, for example in the derivation of a stem cell line. Suitably, the embryo may be cultured to the blastocyst stage.

The subject embryonic or stem-like cells may be used to obtain any desired differentiated cell type, by fusing donor cells with an enucleated oocyte, obtaining embryonic or stem-like cells as described above, and culturing such cells under conditions which favor differentiation to the desired cell type. Exemplary differentiated cells include hematopoietic stem cells and neural cells for the treatment of AIDS, leukemia, Parkinson's disease, Alzheimer's disease, ALS and cerebral palsy, among others.

Double Nuclear Transfer

An embryo resulting from a NT process can be manipulated in a variety of manners. The invention relates to cloned embryos, cells, cell lines, fetuses, and animals that arise from at least one nuclear transfer. Two or more NT procedures may be performed to enhance nuclear transfer efficiency of totipotent embryo, fetus, and animal production and/or placental development. Incorporating two or more NT cycles into methods for cloned embryos, fetuses, and animals can provide further advantages. For example, incorporating multiple NT procedures provides a method for multiplying the number of cloned embryos, fetuses, and animals. Moreover, gene targeting methods require that both copies of a given gene in a diploid cell be targeted in order to knock out or replace the gene. Such methods may require two or more NT procedures in order to efficiently target the gene. The skilled artisan will understand that the methods required for such manipulations will vary, depending on the species of interest.

For NT techniques that incorporate two or more NT cycles, one or more of the NT cycles may be preceded, followed, and/or carried out simultaneously with an activation step. As defined previously herein, an activation step may be accomplished by electrical and/or non-electrical means as defined herein. An activation step may also be carried out at the same time as a NT cycle (e.g., simultaneously with the NT cycle) and/or an activation step may be carried out prior to a NT cycle. Cloned embryos resulting from a NT cycle can be (1) disaggregated or (2) allowed to develop further.

### EXAMPLES

Materials and Methods

Establishment of Cell Lines

Cattle processed at a USDA certified slaughterhouse were used to develop primary cell lines. Tissue samples were taken from various sample sites (kidney, forelimb, intercostals regions, etc.) either: 1) following slaughter but just prior to the carcass being placed in a cooler, 2) following 24 hours at −2° C. or 3) following 24 hours at −2.0° C. and 24 additional hours at 2-4.0°. Tissue was removed from the carcass and placed in PBS+10.0% (v:v) penicillin/streptomycin (10.000 U/ml penicillin G, 10.000 μg/ml streptomycin, Sigma) on ice for transport to the cell culture laboratory. The tissue samples were dissected into small pieces, and tissue explants were cultured in Dulbecco's Modified Eagle's medium (DMEM) F-12 (Sigma) supplemented with 10.0% fetal bovine serum (FBS, Bio Whitaker Inc.) and 1.0% (v:v) penicillin/streptomycin at 37.0° C. in 35 mm tissue culture plates in a humidified atmosphere of 5.0% CO₂ and air. Following establishment of the cell culture, the explants were removed, and cells were harvested by trypsinization and seeded in 75 cm² tissue culture flasks. When the cells reached confluency, they were collected by trypsinization and frozen in DMEM-F12 supplemented with 20.0% FBS and 10.0% dimethyl sulfoxide (Sigma). After thawing, cells were cultured (DMEMI F12 supplemented with 10.0% FBS and 1.0% (v:v) penicillin/streptomycin) to approximately 80% confluence and 15 μM roscovitine (Sigma) was added approximately 24 hours prior to nuclear transfer. Following roscovitine treatment, cells were trypsinized and prepared for nuclear transfer. Further nuclear transfer protocol are discussed below.

Recipient Cytoplasm Preparation

In vitro maturation of bovine oocytes and enucleation is performed as described previously in Cibelli, et al., Cloned transgenic calves produced from non-quiescent fetal fibroblasts, Science 1998, 280: 1256-1258; Wells, et al., Production of cloned calves following nuclear transfer with cultured adult mural granulosa cells, Biol Reprod 1999, 60: 996-1005; and Arat, et al., Production of transgenic bovine embryos by transfer of transfected granulosa cells into enucleated oocytes, Mol Reprod Dev 2001, 60: 20-26. Briefly, bovine cumulus-oocyte complexes (COCs) are recovered by aspiration of antral follicles (3 to 8 mm diameter) on ovaries obtained from a slaughterhouse. Only COCs with a compact, nonatretic cumulus oophorus-corona radiata and a homogenous ooplasm are selected. Oocytes are matured in TCM 199 (Gibco Inc, Grand Island, N.Y.) supplemented with 10% FBS, 50 μg/ml sodium pyruvate, 1% v:v penicillin/streptomycin (10.000 U/ml penicillin G, 10.000 μg/ml streptomycin), 1 ng/ml rIGF-1 (Sigma), 0.01 U/ml bLH and 0.01 U/ml bFSH (Sioux Biochem. Sioux Center, Iowa). Maturation is performed in four-well plates overlaid with mineral oil at 39° C. in a humidified 5% CO₂ in air for 16-18 h. After maturation, the cumulus-corona is removed by vortexing COCs in TL Hepes medium containing 100 U/ml hyaluronidase (Sigma). Maturated oocytes are enucleated with a 15 pin (internal diameter) glass pipette (Eppendorf, Westburg, N.Y.) by aspirating the first polar body and MII plate in a small volume of surrounding cytoplasm. The oocytes are previously stained in TL Hepes containing 2 μg/l ml Hoechst 33342 and 7.5 μg/ml Cytochalasin B (Sigma) for 10-15 min and then kept in TL Hepes supplemented with 7.5 μg/ml Cytochalasin B during enucleation. Enucleation is performed under ultraviolet light to ensure removal of oocyte chromatin.

Donor Cell Preparation and NT

Donor cells are cultured with 10% FBS and allowed to confluency (G1/G0). Immediately before donor cells are transferred into the enucleated oocytes, the cells are dissociated by trypsinization with 0.25% trypsin-EDTA solution (Sigma). The cells are pelletted and resuspended in DMEM F-12+10% FBS. A single cell is inserted into the perivitelline space of the enucleated oocyte by using a 15-μm (internal diameter) glass pipette as described in Cibelli, et al. (1998) (supra). For transfer, the brightest cells in each transgenic cell line are selected under UV light using the FITC filter set. Oocyte-cell complexes are placed in TCM 199 containing 10% FCS at 39° C. in 5% CO₂ in air until fusion.

Fusion and Activation of Oocyte-Cell Complexes

Oocyte-cell complexes are fused by using a needle-type electrode as described in Arat, et al. (2001) (supra); and Miyoshi, et al., Establish of a porcine cell line from in vitro-produced blastocysts and transfer of the cells into enucleated oocytes, Biol Reprod 2000, 62: 1640-1646, in Zimmermann's fusion medium. Zimmermann, et al., Electric field-induced cell-to-cell fusion, J. Membr Biol, 1982, 67: 165-182. The single cell-oocyte couple is sandwiched between two wires arranged in a straight line and attached to micromanipulators. The contact surface between the cytoplast and the donor cell is perpendicular to the electrodes. The distance between the electrodes is approximately 150 μm (the diameter of the oocyte). A single direct current pulse of 40 V for duration of 20 μsec is applied using an LF 101 Fusion Machine (TR Tech Co, Tokyo). Following the pulse, the complexes are cultured in TCM 199 supplemented with 10% FBS for 2 hrs and fusion rates are determined. Activation of NT units is performed as described previously in Arat, et al. (2001) (supra); and Lui, et al., Parthenogenetic development and protein patterns of newly maturated bovine oocytes after chemical activation, Mol Reprod Dev 1998, 49: 298-307, after modification. Briefly, 2 hrs after fusion, nuclear transfer oocytes are exposed to 5 μM calcium ionophore for 10 min (A23187, Sigma), followed by incubation in TCM 199, supplemented with 10% FBS, 2.5 μg/ml Cytochalasin D (Sigma), 10 μg/ml Cycloheximide (Sigma) for 1 hr at 39° C. in 5% CO₂ in air and in TCM 199 with 10% supplemented FBS and 10 μg/ml Cycloheximide for 5 hrs at 39° C. in 5% CO₂, 5% O₂ and 90% N₂.

In Vitro Culture of NT Embryos.

After activation, NT oocytes are cultured in 50 μl culture drops of BARC medium containing bovine serum albumin as described in Arat, et al. (2001) (supra); and Powell, et al., Effects of fibroblast source and tissue-culture medium on success of bovine nuclear transfer with transfected cells, Theriogenology 2001, 55(1): 287, placed into 60 mm culture plate overlaid with mineral oil at 39° C. in 5% CO₂, 5% O₂ and 90% N₂ for 5 days and cleaved NT embryos are transferred into 50 μl culture drops of BARC+BSA medium containing 5% FBS and cultured for an additional 2 days.

Examination of Ploidy and Cell Number of Blastocysts

Blastocysts at Day 7 are cultured in culture medium containing 0.04 μg/ml democolcine (Sigma) and 200 μg/ml heparin (Sigma) for 2.5 hrs. After this incubation period, blastocysts are treated with 0.5% sodium citrate (38° C.) in dH₂O for 4 min and then treated with cold methanol, acetic acid, water (v/v, 3:2:1) for 15-30 sec and placed on a slide. The slides are dried at room temperature for 1 hr and stained with 5% Giemsa solution for 5 min. At least 6 metaphase spreads/blastocyst are counted under a light microscope at 1000× magnification. To examine cell number, 5-10 blastocysts/cell line are counted. Blastocyst stage embryo nuclei are stained on slides in a PBS solution and 10% glycerol containing 1 mg/ml of Hoechst 33342. A drop (˜20 μl) of staining solution containing 1-3 embryos is placed in the center of a slide and a cover slip is placed over the drop and the edges sealed. Nuclei are visualized and counted using a UV light.

Results

TABLE 1
Embryo Development and Pregnancy Data for Production of Cooled Carcass Clones.

| | | | Fused | Cleaved | Blasts | | | Pregnancies | |
|---|---|---|---|---|---|---|---|---|---|
| Session | Donor | Tissue | (%) | (%) | (%) | | ETs | Initial | Ongoing |
| 1 | Y114 | Connective | 39 (72.2) | 17 (44.7) | 7 | (18.0) | 3 | 1 | 0 |
| 2 | Y114 | Kidney | 38 (71.6) | 20 (52.6) | 2 | (5.3) | 1 | 1 | 1 |
| 3 | Y89 | Kidney | 39 (66.1) | 20 (51.2) | 4 | (10.3) | 3 | 2 | 1 |
| 4 | Y114 | Kidney | 57 (78.1) | 44 (77.2) | 16 | (28.1) | 3 | 3 | 0 |

### CONCLUDING REMARKS

Throughout this specification the word ‘comprise,’ or variations such as ‘comprises’ or ‘comprising,’ will be understood to imply the inclusion of a stated element, integer or step, or group of elements, integers or steps, but not the exclusion of any other element, integer or step, or group of elements, integers or steps.

It will be understood that the descriptor “a” is meant to include plural referents unless the context specifically requires otherwise. Thus, in the practice of this invention, providing a cell culture allows for the provision of multiple cell cultures. Similarly, preferential selection of an oocyte from a cell culture allows for the preferential selection of a plurality of oocytes from the cell culture.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1) A method of producing a cloned or genetically modified mammalian non-human embryo comprising:
a) transferring DNA from a donor cell derived from post-mortem non-human mammalian tissue to an oocyte to form a nuclear transfer unit; and
b) culturing said nuclear transfer unit to establish said embryo.

2) A method of producing a cloned or genetically modified mammalian non-human embryo comprising:
a) providing two or more post-mortem tissue samples from two or more different animals;
b) screening the samples for pre-selected physical, genetic and/or phenotypic criteria;
c) transferring DNA from one or more donor cells derived from one or more of the animals to an oocyte to form a nuclear transfer unit; and
d) culturing said nuclear transfer unit to establish said embryo.

3) A method of improving the genotypical, phenotypical, or physical characteristics of a herd of animals comprising:
a) transferring DNA from a first donor cell derived from post-mortem non-human mammalian tissue to a first oocyte to form a first nuclear transfer unit, and
b) culturing said first nuclear transfer unit to establish a first embryo;
c) transferring said first embryo into a recipient female so as to produce a first fetus that undergoes full fetal development and parturition to generate a first live-born animal;
d) mating said first live-born animal with one or more animals of the herd.

4) The method of claim 1 or 2, further comprising transferring said embryo into a recipient female so as to produce a fetus that undergoes fall fetal development and parturition to generate a live-born animal.

5) The method of claim 1, 2, or 3, wherein said animal is a cow.

6) The method of claim 1, 2, or 3, wherein said animal is a pig.

7) The method of claim 1 or 2 wherein said non-human embryo is capable of developing into a live-born animal.

8) The method of claim 1, 2, or 3, wherein said tissue has been cooled to about 10° C. or less.

9) The method of claim 1, 2, or 3, wherein said tissue has been cooled to about 0° C. or less.

10) The method of claim 1, 2, or 3, wherein said tissue is at least about 2 hours post-mortem.

11) The method of claim 1, 2, or 3, wherein said tissue is at least about 30 hours post-mortem.

12) The method of claim 2 wherein at least about 10 samples are screened for the pre-selected criteria.

13) The method of claim 2 wherein a least about 50 samples are screened for the pre-selected criteria.

14) The method of claim 2 wherein said tissue samples are derived from at least 5 herds of animals.

15) The method of claim 2 wherein said donor cell is derived from tissue different from which the tissue sample is derived.

16) The method of claim 2 wherein said tissue is screened for quality.

17) The method of claim 2 wherein said tissue is selected from adipose and muscular tissue.

18) The method of claim 2 wherein said tissue has been scored prime or higher.

19) The method of claim 2 wherein said tissue is screened for genetic characteristics.

20) The method of claim 3 further comprising:
a) transferring DNA from a second donor cell derived from post-mortem non-human mammalian tissue to a second oocyte to form a second nuclear transfer unit;
b) culturing said second nuclear transfer unit to establish a second embryo;
c) transferring said second embryo into a recipient female so as to produce a second fetus that undergoes full fetal development and parturition to generate a second live-born animal;
d) mating said second live-born animal with one or more animals of the herd.

21) The method of claim 20 wherein the first donor cell and the second donor cell are derived from the same tissue.

22) The method of claim 20 wherein the first donor cell and the second donor cell are derived from different tissue.

23) The method of claim 20 wherein the first donor cell and second donor cell are derived from different genders.

24) The method of claim 20 wherein the first donor cell and second donor cell are derived from different genders, and the first live born animal and second live born animal are mated with one or more animals of the herd or with each other.

25) The method of claim 20 wherein:
a) the first donor cell and second donor cell are derived from different genders,
b) the first donor cell and second donor cell share a substantially identical genotype, phenotype, or physical characteristic, and
c) the first live born animal and second live born animal are mated.

27) A method of producing cattle in a cattle breeding operation comprising:
a) providing a herd comprising a plurality of female cattle and a male stud,
b) mating the female cattle and the male stud to produce a plurality of male and female offspring,
c) cloning a progenitor male or female offspring from the plurality of male and female offspring to produce a clone,
d) replacing the stud bull or one cow of said female cattle with said clone, and
e) repeating step b) one or more times to produce a plurality of improved male and female offspring.

28) The method of claim 27 wherein:
a) said progenitor male offspring is cloned and the male clone replaces the stud bull, and
b) the female cow that birthed the progenitor male offspring is removed from the herd.

29) The method of claim 27 wherein said progenitor male offspring is cloned and the male clone replaces the stud bull further comprising:
f) cloning the progenitor male offspring a second time to produce a second male clone;
g) providing a second herd comprising a second plurality of female cattle and a second male stud;
h) replacing the second male stud with said second male clone.

30) The method of claim 27 wherein said progenitor male offspring is cloned and the male clone replaces the stud bull, further comprising:
f) cloning an improved progenitor male offspring from said improved male and female offspring to produce an improved male clone,
g) replacing the male stud with said improved male clone, and
h) repeating step b) one or more times to produce a plurality of twice improved male and female offspring.

31) The method of claim 27 wherein:
a) said progenitor female offspring is cloned and used to replace the one cow; and
b) said male stud is replaced.

32) The method of claim 27 wherein said progenitor female offspring is cloned and used to replace the one cow further comprising:
a) cloning the progenitor female offspring a second time to produce a second female clone; and
b) replacing a second female cow in said herd with said second female clone.

33) The method of claim 27 wherein said progenitor female offspring is cloned and used to replace the one cow further comprising:
f) cloning a second progenitor female offspring to produce a second female clone; and
g) replacing a second female cow in said herd with said second female clone.

34) The method of claim 27 wherein said progenitor female offspring is cloned and used to replace the one cow further comprising:
f) cloning an improved progenitor female offspring from said improved male and female offspring to produce an improved female clone;
g) replacing a female cow in said herd with said improved female clone; and
h) repeating step b) one or more times to produce a plurality of twice improved male and female offspring.

35) The method of claim 27 wherein said progenitor female offspring is cloned and used to replace the one cow further comprising:
f) cloning the progenitor female offspring a second time to produce a second female clone;
g) providing a second herd comprising a second plurality of female cattle and a second male stud;
h) replacing a female cow in said second herd with said second female clone.

36) The method of claim 27 wherein said progenitor male offspring is cloned and used to replace the stud bull, further comprising:
f) cloning a progenitor female offspring to produce a female clone,
g) providing a second herd comprising a second plurality of female cattle and a second male stud; and
h) replacing a female cow in said second herd with said second female clone.

37) The method of claim 27 further comprising, before step c), castrating said male offspring.

38) The method of claim 27 further comprising, before step c), sacrificing said male and female offspring.

39) The method of claim 27 wherein said mating is via artificial insemination.
